(19) United States
(12) Patent Application Publication
(10) Pub. No.: US 2001/0056073 A1
(43) Pub. Date: Dec. 27, 2001

(54) GENE THERAPY METHOD FOR REVASCULARIZING ISCHEMIC TISSUE

(76) Inventors: ASCHER SHMULEWITZ, SEATTLE, WA (US); JERRY SANDERS, SAN FRANCISCO, CA (US)

Correspondence Address: DAVIS WRIGHT TREMAINE, LLP, 2600 CENTURY SQUARE, 1501 FOURTH AVENUE, SEATTLE, WA, 98101-1688, US

(21) Appl. No.: 09/120,493

(22) Filed: Jul. 21, 1998

(51) Int. Cl. A61K 48/00

(52) U.S. Cl. 514/44

(57) ABSTRACT

There is disclosed a method for optimizing revascularizing ischemic tissue, particularly ischemic myocardial tissue. Specifically, there is disclosed a means for retrograde perfusion of angiogenic gene therapy vectors to ischemic tissue, wherein the polypeptide(s) encoded by the gene therapy vectors promote angiogenesis and neovascularization, and are selected from the group consisting of VEGF, ICAM-1, FGF, EGF, nitric acid synthase (NOS), E-selectin, and combinations thereof.

### TECHNICAL FIELD OF THE INVENTION

The present invention provides a method for optimizing revascularizing ischemic tissue, particularly myocardium. Specifically, this invention provides a means for retrograde perfusion of gene therapy vectors that encode angiogenic growth factors to ischemic tissue, wherein the polypeptide(s) encoded by the gene therapy vectors promote angiogenesis and neovascularization.

### BACKGROUND OF THE INVENTION

Patients with end-stage ischemic cardiomyopathy have no treatment options other than heart transplantation or a different organ. There are various temporary measures to address symptoms, such as a device that would provide a short conduit between the left ventricular cavity of the heart and the coronary sinus to provide a device means for retrograde perfusion of ischemic myocardium. This retrograde flow is maximal during systole to enhance the insufficient antegrade diastolic coronary arterial flow into the same myocardial tissue beds.

Retrograde coronary sinus perfusion is used routinely to deliver cardioplegia during cardiac surgery. There have been two problems associated with retrograde coronary sinus perfusion, including a lack of adequate perfusion through the coronary system to the right ventricle and frequent myocardial edema.

During an immune response, endothelial cells transiently up-regulate genes encoding cell adhesion molecules (VCAM-1, ICAM-1 and E-selectin) and chemotactic cytokines that mediate the interaction of the endothelium with circulating leukocyte cells. Antibody blocking of VCAM-1 or ICAM-1 leukocyte adhesion receptors inhibited the development of transplant arteriopathy in rabbit models. However, such antibodies are not therapeutically useful because such antibodies to leukocyte adhesion receptors leave circulating leukocytes unable to respond to infectious challenges elsewhere in the body. Therefore, it is important to target only the endothelial adhesion molecule expression in cardiac endothelium to treat on the target organ and not the hosts leukocytes.

Gene expression of both adhesion molecules and cytokines is mediated predominantly at the transcriptional level and often involves members of the NF-kappaB (NFκB)/Rel protein family of transcription factors. Growth factors, such as vascular endothelial growth factor (VEGF), endothelial growth factor (EGF) and fibroblast growth factor (FGF), which might be necessary for vessel repair after injury, are regulated primarily through different transcription factors. NFκB has been implicated in the signal transduction that prevents cell death elicited by the cytokine TNFα. Thus, it is difficult to block NFκB genetically without increasing a cells susceptibility to apoptotic stimuli.

Several attempts have been made to partially block NFκB while still allowing for protection from apoptosis. Some anti-arthritis drugs, such as glucocorticoids, salicylates and gold compounds can partially block NFκB. Thus, only partial reduction of NFκB activation may be sufficient to reduce the production of inflammatory cell adhesion molecules and cytokines to a significant degree, while preserving endothelial cell viability.

Both over expression of nitric oxide synthase (NOS) and a mutated dominant negative IκB have both been shown to block NFκB activation and endothelial adhesion molecule expression in vitro. Augmenting NO levels has been reported to suppress neointimal formation after vascular injury. Both liposomal and adenoviral vectors have been used for delivery of gene therapy vectors. Therefore, there is a need in the art to develop a means for delivering various gene therapy vectors to appropriate endothelial cells only in those vascular beds requiring expansion, such as ischemic myocardial tissue.

### SUMMARY OF THE INVENTION

The present invention provides a method for revascularizing ischemic tissue, such as myocardium, comprising administering an angiogenesis inducing gene encoding an angiogenesis growth factor encapsulated in a liposomal carrier vehicle into tissue and targeted to vascular endothelial cells. Preferably, a means for administering the angiogenesis inducing gene encoding an angiogenesis growth factor for ischemic myocardial tissue, is into the coronary circulation. Most preferably, the means for administration into the coronary circulation or any circulation of ischemic tissue is through retrograde perfusion of the vasculature, particularly the coronary vasculature. Preferably, the angiogenesis growth factor is selected from the group consisting of VEGF, ICAM-1, FGF, EGF, nitric acid synthase (NOS), E-selectin, and combinations thereof. Preferably, the means for retrograde perfusion of the myocardium is by a catheter inserted into a patient's regional coronary venous system supplied into that region of ischemic myocardium, wherein the catheter has an inflatable balloon containing the liposomally encapsulated gene vectors.

### BRIEF DESCRIPTION OF THE DRAWINGS

FIG. 1 shows a photograph from sections in example 1 showing a distribution of liposome gene therapy vehicle uptake by coronary endothelial cells in a rabbit heart after a 10 minute exposure to liposomes. These data indicate that the gene carried by liposomal gene therapy vehicles are likely to be transfected into coronary artery endothelial cells as the proper target for the inventive method. In this experiment, the liposomes were complexed to the lipid label Di-D and delivered to the coronary vasculature by aortic root injection.

FIG. 2 shows Di-D-labeled liposome adsorption to endothelial cells following a 10 minute exposure to the complexed liposomes.

FIG. 3 shows a frequency distribution of differing DNA to lipid liposome ratios based upon transfection efficiency as measured by expression of an alkaline phosphatase reporter gene 24 hours after transfection.

FIG. 4 shows alkaline phosphatase reporter gene product expression in carotid artery preparations for control transfections (i.e., no reporter gene construct) and 6 or 24 hours after transfection. These data show the presence of the reporter gene product as early as 6 hours after transfection with as little as a 10 minute exposure and much greater reporter gene product expression 24 hours after expression.

FIG. 5 shows NFκB activation in control (L) and transplanted (R) hearts at 24 hours. The two photographs visualize NFκB by an antibody to the p65 epitope which can be exposed only after nuclear translocation. These data indicate that due to the low cell cycle activity of myocardial tissue, gene transfection and translocation into the nucleus of myocardial tissue results in long and stable gene integration and longer term expression of the gene product.

FIG. 6 shows a diagram of the in situ rabbit carotid artery segment model without transplantation. This diagram shows where the liposomes containing the gene of interest were added into the lumen of a clamped carotid artery segment for temporary administration of a gene therapy product.

FIG. 7 shows a perspective view of the human heart, partly in section, illustrating implantation of an delivery device for retrograde delivery into the coronary vasculature of oxygenated blood and the liposomally-encapuslated gene for transfection of vascular endothelial cells located primarily in the coronary vasculature in general and in areas of ischemic tissue in particular.

FIG. 8 is a perspective view of the retrograde perfusion apparatus of FIG. 7.

FIG. 9 is a sectional view of a first conduit of the preferred retrograde perfusion device for delivering the inventive pharmaceutical formulation to the coronary vasculature.

FIG. 10 is a sectional view of a second conduit of the preferred retrograde perfusion device for delivering the inventive pharmaceutical formulation to the coronary vasculature.

FIG. 11 is a perspective view of a human heart, partly in section, illustrating implantation of a second preferred delivery device for retrograde delivery into the coronary vasculature of oxygenated blood and the liposomally-encapuslated gene for transfection of vascular endothelial cells located primarily in the coronary vasculature in general and in areas of ischemic tissue in particular.

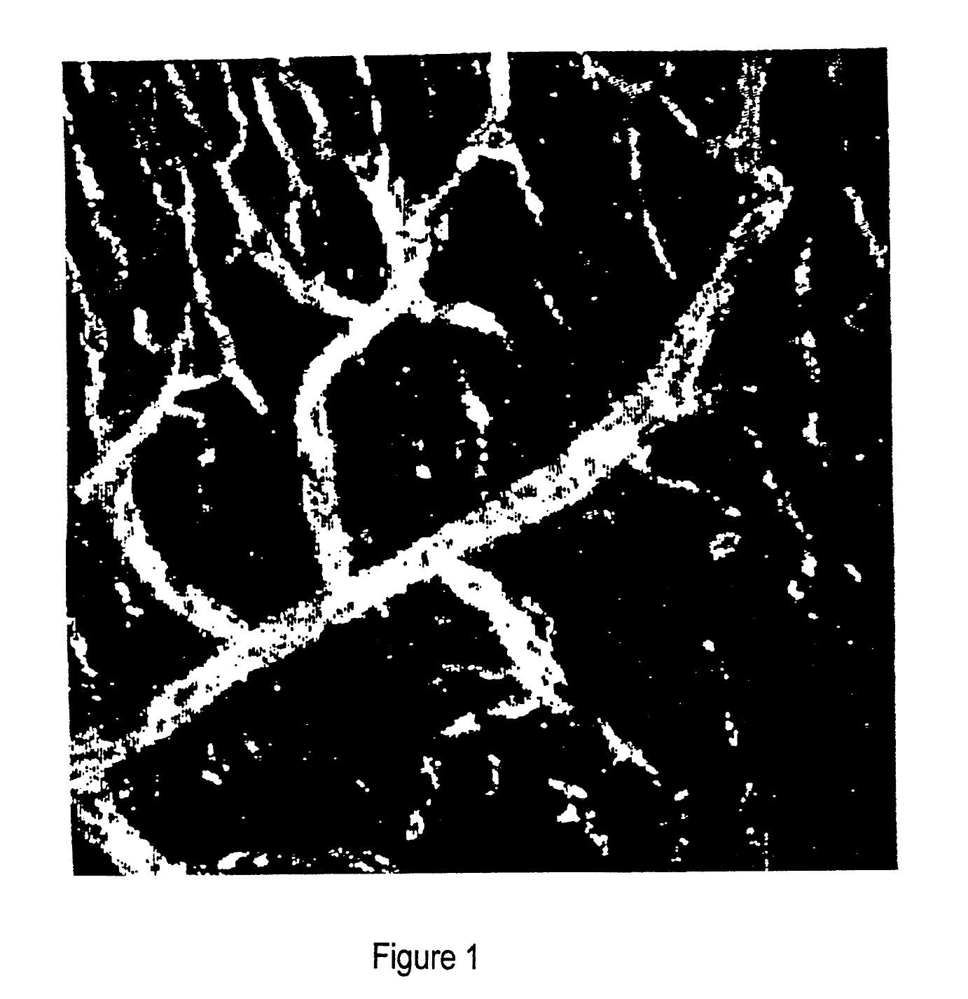

### DETAILED DESCRIPTION OF THE INVENTION

Device for Retrograde Delivery of Gene Therapy Vectors to Ischemic Tissue

The inventive method for revascularizing ischemic tissue can utilize any retrograde perfusion device having a reservoir means for storing liposomally-encapsulated angiogenic growth factor gene sequences operably linked to an expression vector. A preferred delivery apparatus for ischemic myocardial tissue, for example, provides a means for draining a volume of blood from the left atrium or ventricle of the heart and directing that blood into the coronary venous vasculature to provide retrograde perfusion to the myocardium and further provide a means for mixing the inventive liposomally-encapsulated angiogenic growth factor gene sequences (inventive pharmaceutical formulation) to the ischemic myocardial tissue. Such an apparatus, for example, comprises a first conduit having an inlet end configured for transluminal insertion into a patient's left atrium or left ventricle, and coupled to a second conduit having an outlet and configured for transluminal insertion into the coronary venous vasculature via the coronary ostium. A pump means (which may be motor-driven, hydraulically actuated or the beating heart) is coupled to a fluid circuit formed by the first and second conduits and a reservoir means containing the inventive pharmaceutical formulation, to cause infusion of oxygenated blood (from the left atrium or left ventricle) containing the pharmaceutical formulation (from a reservoir means) into the coronary venous vasculature. The coronary ostium may be either partially or fully occluded by the outlet of the second conduit. The pump may also be operated with a duty cycle to control a parameter related to the pressure in the coronary venous system, so as to reduce the potential for edema of the venous system. The fluid circuit provides a pressure gradient sufficient to cause flow to the coronary venous system to improve hypoxia with oxygenated blood and provide a means for delivering the inventive pharmaceutical formulation to those areas of ischemic tissue that would otherwise be difficult to infuse. Moreover, it is important that the inventive pharmaceutical formulation be delivered to vascular endothelial cells located in ischemic tissue so as to improve the vasculature in such tissue and generate an angiogenic response in that tissue most in need of improved angiogenesis.

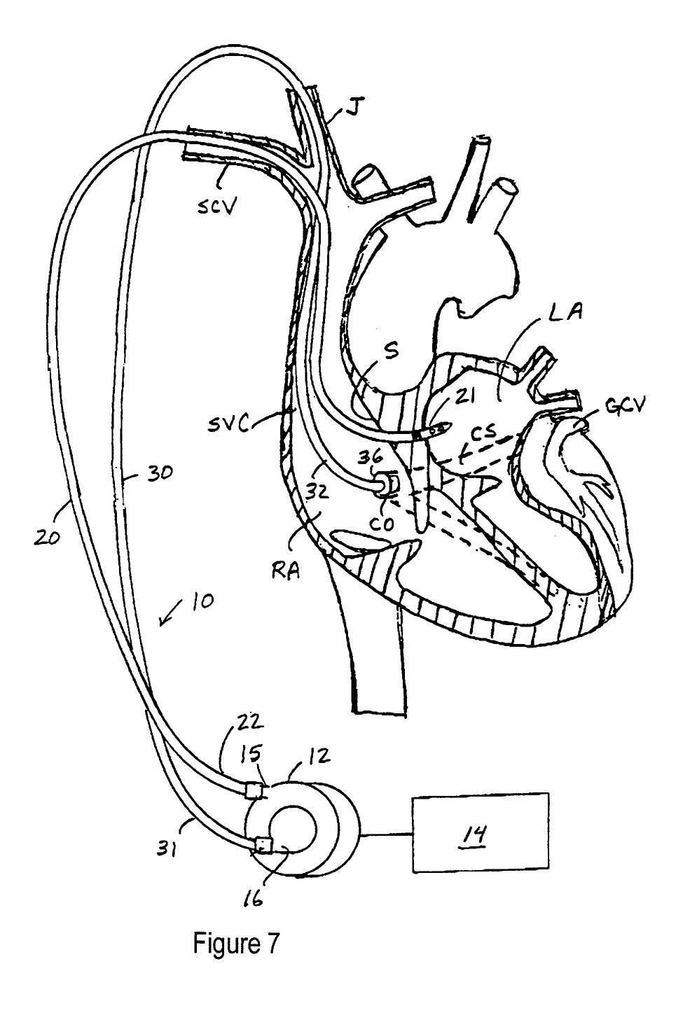

Referring to FIGS. 7-10, a first embodiment of inventive pharmaceutical formulation delivery apparatus 10 comprises conduits 20 and 30 coupled to a motor-driven pump 12. Pump 12 includes an inlet port 15 an outlet port 16 and can be a commercially available infusion pump or centrifugal pump. Control circuitry 14 controls operation of pump 12. Conduit 20 has an inlet end 21, and outlet end 22 and lumen 23 connecting the inlet and outlet ends (FIG. 9). Inlet end 21 may be transluminally inserted (via the right jugular vein J or the right subclavian vein SCV and superior vena cava SVC into the right atrium RA and extends through a puncture in the atrial septum S into the left atrium LA. Inlet end 21 preferably includes a central opening 24, a plurality of lateral openings 25 and a bullet-shaped of conical-shaped tip 26 that enables the inlet end 21 to be urged along a guide wire to penetrate the atrial septum. The insert end 21 also preferably includes a radio-opaque market band 27 to enable the location of the inlet end to be determined using a fluoroscope. The outlet end 22 is coupled to the inlet 15 of pump 12 by fitting 28.

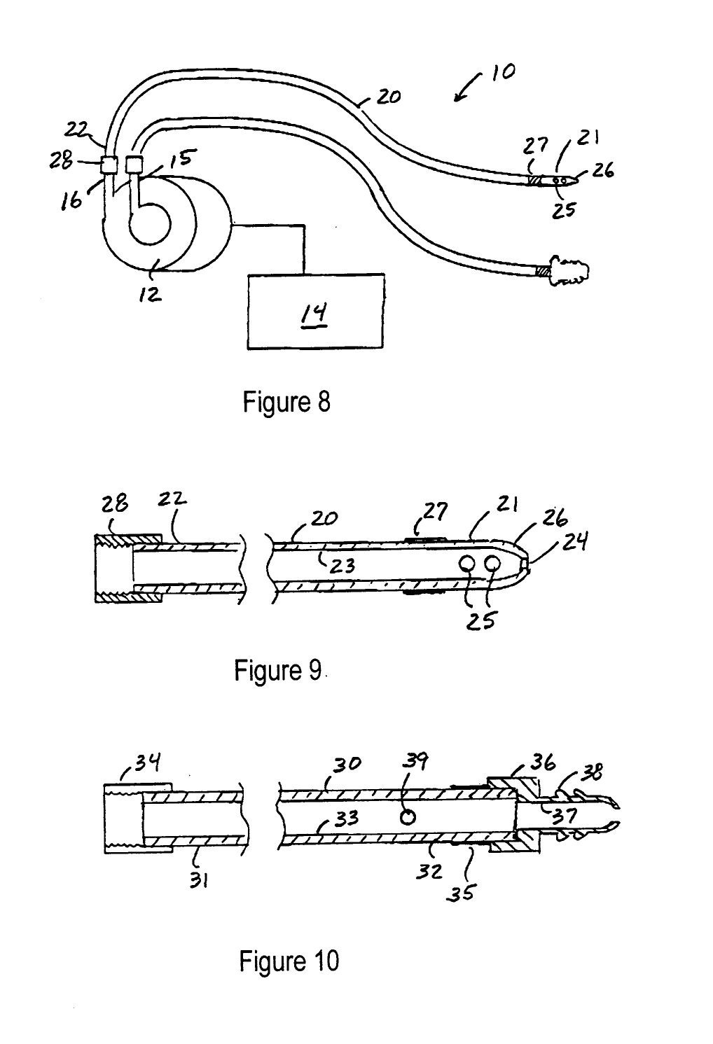

Alternatively, inlet end 21 of conduit 20 may be inserted transluminally and transseptally and then passed through the mitral valve from the left atrium into the left ventricle. Short term use of conduit 20 in this manner should not adversely affect the mitral valve. Alternatively in FIG. 8, inlet end 21 of conduit 20 may be inserted transluminally via the femoral artery and aorta into the aortic root and then passed through the aortic valve into the left ventricle.

Conduit 30 has an inlet end 31, and outlet end 32 and a lumen 33 connecting the inlet and outlet ends (FIG. 10). Inlet end 31 is coupled to outlet port 16 of pump 12 by a fitting 34. Outlet end 32 is transluminally inserted via the right subclavian vein SCV, or right internal jugular vein J, and superior vena cava SVC into the right atrium RA and extends through the coronary ostium CO into the coronary sinus CS. Preferably, outlet end 32 includes a radio-opaque marker band 35 and a plug 36. The plug 36 has a bore 37 and a plurality of barb and rib-type projections 38 that engage the interior wall of the coronary sinus to retain the plug in the coronary sinus until forcibly removed. When inserted into the coronary sinus, outlet end 32 may either partially of fully occlude the coronary ostium CO and permit partial flow from the coronary sinus into the right atrium.

Alternatively, instead of disposing outlet end 32 of conduit 30 in the coronary sinus, outlet end 32 may be advanced through the coronary sinus into another portion of the cardiac venous vasculature, for example the great cardiac vein GCV to provide more localized retroperfusion of the myocardium. In this configuration, plug 36 may be configured so that conduit 30 passes through it a predetermined distance, or plug 36 may be omitted entirely. In addition, conduit 30 may include one or more openings 39 for venting a portion of blood from conduit 30 into the right atrium, for example, when the volume of blood drained from the left atrium or left ventricle to reduce left ventricle exertion is greater than the volume needed to profuse the venous system.

Conduits 20 and 30 are made from a biocompatible flexible material, such as materials used in catheters (e.g., polyvinyl chloride, polyethylene or silicone). Conduit 30 is preferably more rigid than conduit 20, so that plug 36, if present, may be removably seated in the coronary ostium CO by exerting force on inlet end 31 of the conduit. Plug 36 preferably is made from an elastomeric material, such a rubber, latex or silicone.

The device can be implanted for the purposes of delivering the inventive pharmaceutical product by, for example, implanting conduit 20 using a transluminal approach that is a variation of the Brockenbrough method of catheterizing the left ventricle. A conventional Brockenbrough technique employs a catheter needle combination that is advanced through the right femoral artery and into the right atrium and used to puncture the septum between the right and left atria. Thus, one can use a Brockenbrough needle (commercially available) to advance a guide wire into the right atrium via the right internal jugular vein. The Brockenbrough needle punctures the atrial septum and then the puncture is dilated using, for example, progressively larger catheters, which are withdrawn and leaving the guide wire in place.

Conduit 20 is slipped over the proximal end of the guide wire, via central opening 24, so that the guide wire passes through lumen 23 and exists through fitting 28. Conduit 20 is then advanced over the guide wire so that inlet end 21 passes through the transseptal puncture and into the left atrium, as determined by appropriate visual methods. If needed, the inlet end 21 of conduit 20 can be advanced through the mitral valve into the left ventricle. Once the inlet end is positioned, the guide wire is withdrawn proximately through fitting 28. Fitting 28 is coupled to inlet port of pump 12 and hooked up to an additional reservoir containing the liposomally-encapsulated gene vector encoding an angiogenic growth factor pharmaceutical composition for administration.

Again using standard catheterization techniques, a guide wire is inserted transluminally via the right internal jugular vein (or the right subclavian vein) through the superior vena cava and into the coronary sinus via the coronary ostium. Conduit 30 is slipped over the proximal end of the guide wire, via bore 37 in plug 36, so that the guide wire passes through lumen 33 and exits through fitting 34. Conduit 20 is advanced over the guide wire so that plug 36 passes through the coronary ostium and becomes lodged in the coronary sinus. Alternatively, one may advance outlet end of conduit 30 through the coronary sinus into a selected cardiac vein (such as the great cardiac vein). Once outlet end 32 of conduit 30 is positioned in the coronary venous vasculature, the guide wire is withdrawn proximately through fitting 34. Fitting 34 is then coupled to outlet port 16 of pump 12, completing implementation of the liposomally encapsulated drug delivery apparatus for retrograde drug delivery into the coronary vasculature.

Pump 12 further contains a drug reservoir and a drug solution inlet (not shown) that is able to mix liposomally encapsulated gene DNA sequences encoding angiogenic growth factors into oxygenated blood being pumped in retrograde fashion into the coronary vasculature.

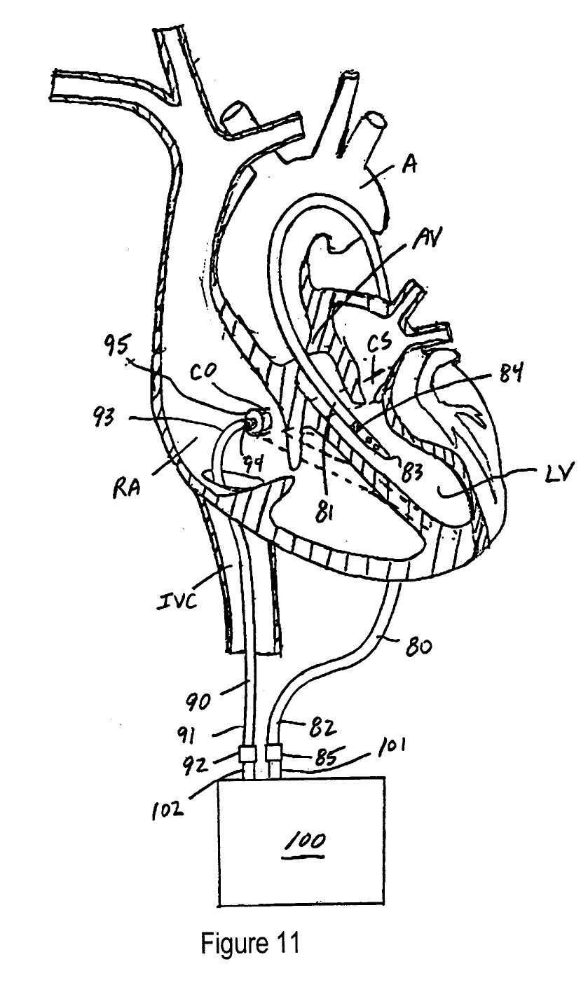

Alternatively and referring to FIG. 11, apparatus 60 comprises conduit 80, conduit 90 and hydraulically-actuated pump 100. Inlet end 81 of conduit 80 is configured to be inserted via a femoral artery and through the aorta A and aortic valve AV into left ventricle LV. Conduit 90 is configured to be inserted via a femoral vein and through inferior vena cava IVC and right atrium RA into the coronary sinus via the coronary ostium CO.

For administering a liposomally-encapsulated gene sequence encoding angiogenic growth factors locally to non-myocardial tissue, the pharmaceutical composition is administered to a particular vascular bed of ischemic tissue. Generally the cause of the ischemia is due to poor circulation of the arterial side, so retrograde administration is preferred. A retrograde flow can be accomplished, for example, by setting up a balloon catheter on venous side draining the vascular bed of the ischemic tissue. Simultaneously, a catheter can be administering the liposomally-encapsulated gene sequence encoding angiogenic growth factors from the tip of the balloon if the arterial side is blocked by some occlusion. If there is still some arterial flow, the proximal (arterial) side flow can be blocked while using collaterial vascular flow through an alternative venous drainage to pump the liposomally-encapsulated gene sequence encoding angiogenic growth factors in a retrograde flow to contact vascular endothelial cells within the vascular of the ischemic tissue.

Angiogenic Nucleotide Sequences for Transfecting Myocardial Endothelial Cells

The angiogenic growth factor gene products used according to the inventive method include, but are not limited to, VEGF, ICAM-1, FGF, EGF, nitric acid synthase (NOS), E-selectin, and combinations thereof. Each human gene sequence is known and has been reported in the scientific literature. The procedures for making the gene constructs utilizing the foregoing angiogenic growth factor gene sequences is standard in the art and is provided, for example, in Samblook et al. Manuel of Molecular Biology Techniques.

Liposomal Gene Vector Delivery

Liposome-mediated transfection provides a noninfectious method for transfecting endothelial cells in the coronary circulation to translate angiogenic growth factors in the local coronary circulation to promote coronary vasculature and allow for better circulation in ischemic myocardial tissue. Liposomal carriers for gene therapy have been successful in vivo (Zhu et al. Science 261:209-211, 1993) for efficient systemic transfection with reporter genes in mice after peripheral venous injection. DNA-liposome complexes are made, for example with cationic and neutral lipid liposomes in a ratio from about 2:1 to about 1:2 mixed with plasmid DNA containing the angiogenic growth factor cDNA sequence operably linked to a mammalian expression vector, such as human cytomegalovirus enhancer and promoter as well as a 5′ intron from the preproinsulin gene (Cullen et al., Nature 307:241-245, 1984), wherein the gene encoding the angiogenic growth factor sequence is followed in phase by, for example, the SV40 early region poly A site. In the examples provided herein, a reporter gene is substituted instead of an angiogenic growth factor gene, however, it is well within the skill of those in the art to add any gene of interest into such a gene construct. The liposome containing the angiogenic growth factor gene sequence in the appropriate expression vector is added to a reservoir means in an appropriate myocardial retrograde perfusion device in a pharmaceutically-acceptable carrier, such as 5% dextrose in water.

The inventive method was first tested in predictive donor heart models in an ex vivo model. The predictive models use reporter genes, instead of angiogenic growth factor genes, to determine optimal concentrations, vector constructs, transcription efficiency, liposomal adsorption efficiency and timings of appearance of gene products to provide for the predictability of the inventive method using angiogenic growth factor genes (wherein endpoints of gene product transcription and angiogenic effect cannot be easily measured on a quantitative basis).

One concern with gene therapy for increasing myocardial circulation based upon angiogenic growth factors is whether gene products would be produced early enough to affect initial endothelial activation. In a model of transplanted control hearts, transfected VCAM-1 genes were not expressed in rabbit coronaries until 6 hours after transplant when the exposure to the liposome gene therapy product was done ex vivo. Therefore, the appearance of VCAM-1 gene product was due to newly-synthesized expression, after transplantation, and not as a result of ischemia alone prior to transplantation. Moreover, VCAM-1 expression was noticed at 6 hours after transplantation, but its expression in coronary arteries and veins was not widespread until 24 hours after transplantation. These data indicate that gene product expression to the coronary vasculature will parallel adhesion molecule expression following ischemia and reperfusion.

Other Gene Therapy Delivery Means

In addition to liposomal gene therapy delivery means, one can also use viral vectors for delivery of angiogenesis-inducing genes encoding angiogenesis growth factors. One example is adenoviral transduction of endothelial cells. For example, an adenoviral construct was constructed using a CMV promoter and an alkaline phosphatase reporter gene to transduce cultured rabbit arterial endothelial cells in culture. Duplicate wells were grown to confluency (10⁵ cells/well) and then transduced with 10, 30, 100, 300 or 1000 virons per cell (multiplicity of infection or MOI). After 18 hours, a few cells expressed reporter gene product activity at an MOI of 10 and there was a progressive increase in the percentage of cells transduced with increasing MOI numbers, until an MOI of 300 when virtually all of the cells expressed the gene product. The cells that were exposed to 1000 MOI appeared darker than others, suggesting more alkaline phosphatase copies per cell.

Therefore, standard viral vectors currently employed in general systemic gene therapy procedures, can be modified with a construct containing an angiogenesis-inducing gene encoding an angiogenesis growth factor and used for local myocardial delivery to the coronary vasculature. Preferably, the means for delivery to the coronary vasculature is by retrograde perfusion of balloon angioplasty means.

### EXAMPLE 1

This example illustrates an experiment showing that liposomal gene therapy delivery vehicles will adsorb to endothelial cells. It is important in the development of the inventive method to ascertain that the gene therapy delivery vehicle, a liposomal vehicle, will be adsorbed at the needed site of action when such low levels of therapeutic gene therapy agent is delivered over time to affected ischemic myocardial tissue.

Liposomes were labeled with a red-fluorescent Di-D lipid tracer (Molecular Probes, Eugene Oreg.) according to the manufacturers instructions to form labeled liposomes. Rabbit excised donor hearts were surgically obtained and the aorta was cross-clamped after delivery of D5W-based cardioplegia. The labeled liposomes were complexed to a reporter gene expressing chloramphenicol aceyl transferase (CAT) which can be assayed for enzymatic activity. The complexed liposomes were infused into the aortic root and down coronary arteries. After a 10 minute exposure time, excess complexed liposomes were flushed out with D5W. Aortic root perfusions fixed the myocardial tissue with 1% paraformaldehyde and bovine serum albumin (BSA).

In an effort to further visualize endothelial cells, the aortic root and coronary circulation of the rabbit hearts were secondarily perfused win an FTIC-labeled pan-endothelial lecitin. The whole heart was cross-sectioned by a sledge microtome and liposomes and endothelial cells were visualized by confocal microscopy. Photographs taken showed that Di-D-labeled liposomal absorption was accomplished in intramyocardial coronary arteries. These data surprisingly show that liposomal gene therapy vehicles were well and diffusely adsorbed to coronary artery endothelium in donor hearts after only a 10-minute exposure to the liposomal-DNA complexes.

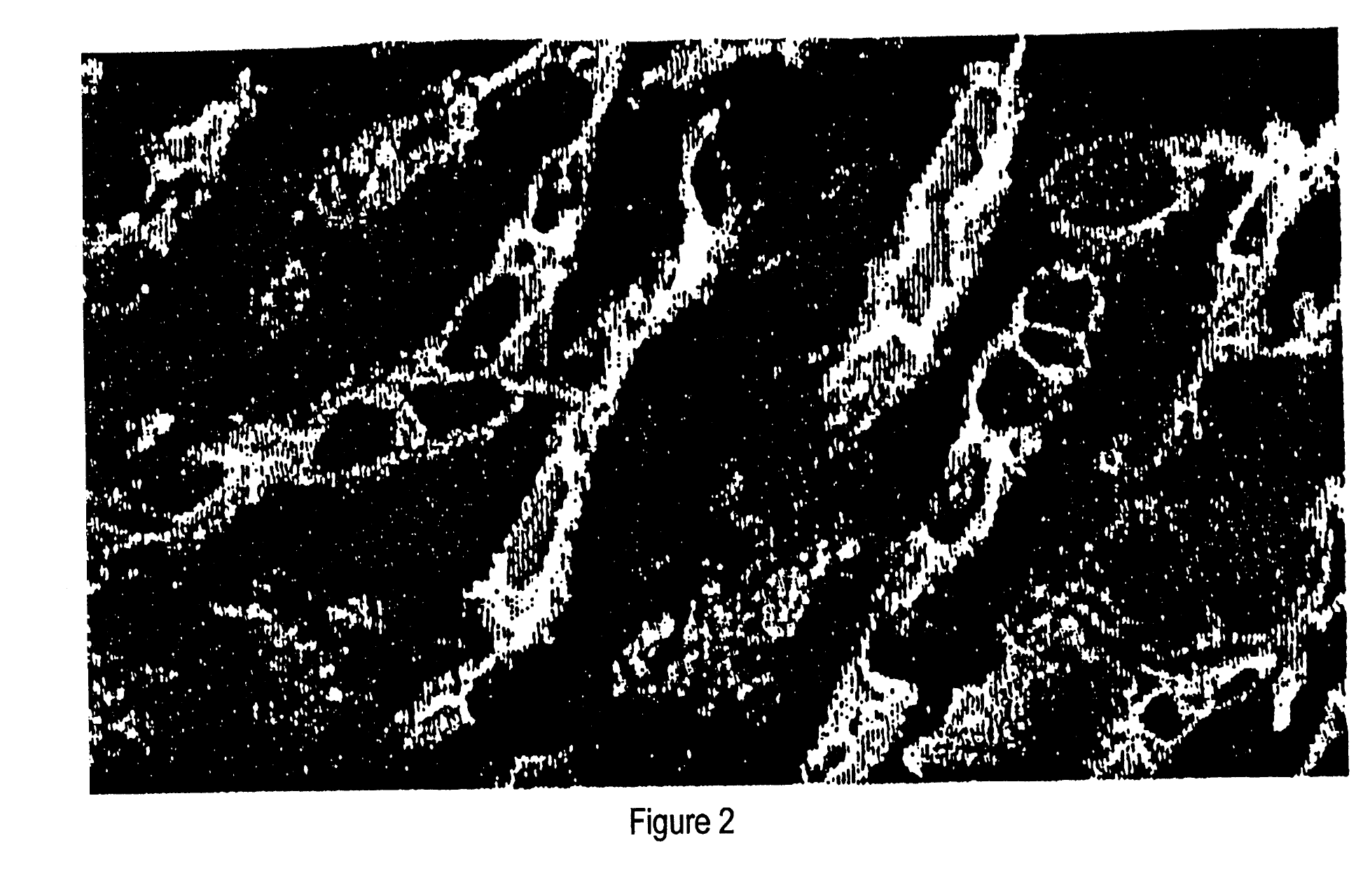

The carotid segment was transiently isolated between vascular clamps to simulate ex vivo treatment of donor hearts, the lumen was filled with D5W, and perfused with the liposome-DNA complexes made in this example for 10 minutes. The liposomal complexes were flushed out after 10 minutes and normal blood flow was resumed. An en face preparation was made of the endothelial surface of these arteries so that the whole vessel lumen, laid out longitudinally, was examined by confocal microscopy. This process examined the efficiency of liposome adsorption in in vivo liposome transfections of untransplanted rabbit carotid arteries. FIG. 2 shows a higher magnification of the endothelial surface of a treated rabbit carotid artery showing that the liposome-DNA complexes were adsorbed to the majority of endothelial cells.

### EXAMPLE 2

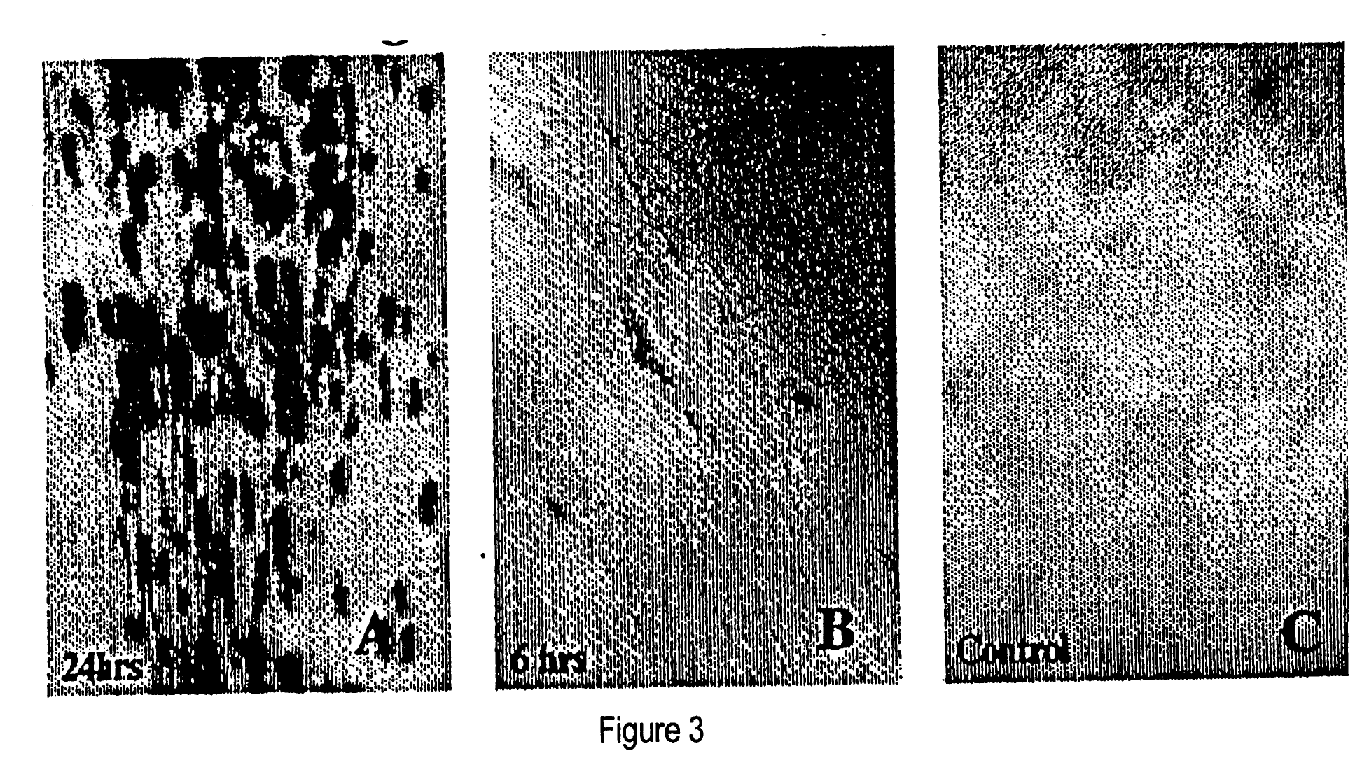

This example illustrates a determination of appropriate DNA and lipid ratios for formation of optimal complexed liposomes. These data provide for efficient angiogenic gene delivery for maximal transfection of myocardial endothelial tissue. Untransplanted rabbit carotid arteries were used as a model of coronary arterial transfection. Four different DNA-lipid ratios were instilled in a blinded fashion with just a coded label into 18 carotid segments. The DNA segment included an alkaline phosphatase reporter gene for visualization by immunocytochemistry. FIG. 3 reports the data using a software product (Optimus, Edmunds, Wash.) to calculate transfection efficiency by area expressing gene product divided by the total area in each of 32 standardized grids per carotid specimen. The software was programmed to identify 32 standard grids on each carotid specimen to eliminate human selection bias.

The ratios are indicated in FIG. 3 as the ratio of DNA to lipid. The data in FIG. 3 show that the most optimal ratio was 1:1 DNA:lipid (on a per weight basis) for optimal arterial endothelial transfection. These data indicate that liposomal transfection uptake is quite high and the prevalence of expression of the reporter gene product indicates the likelihood of success with angiogenic growth factor gene products.

### EXAMPLE 3

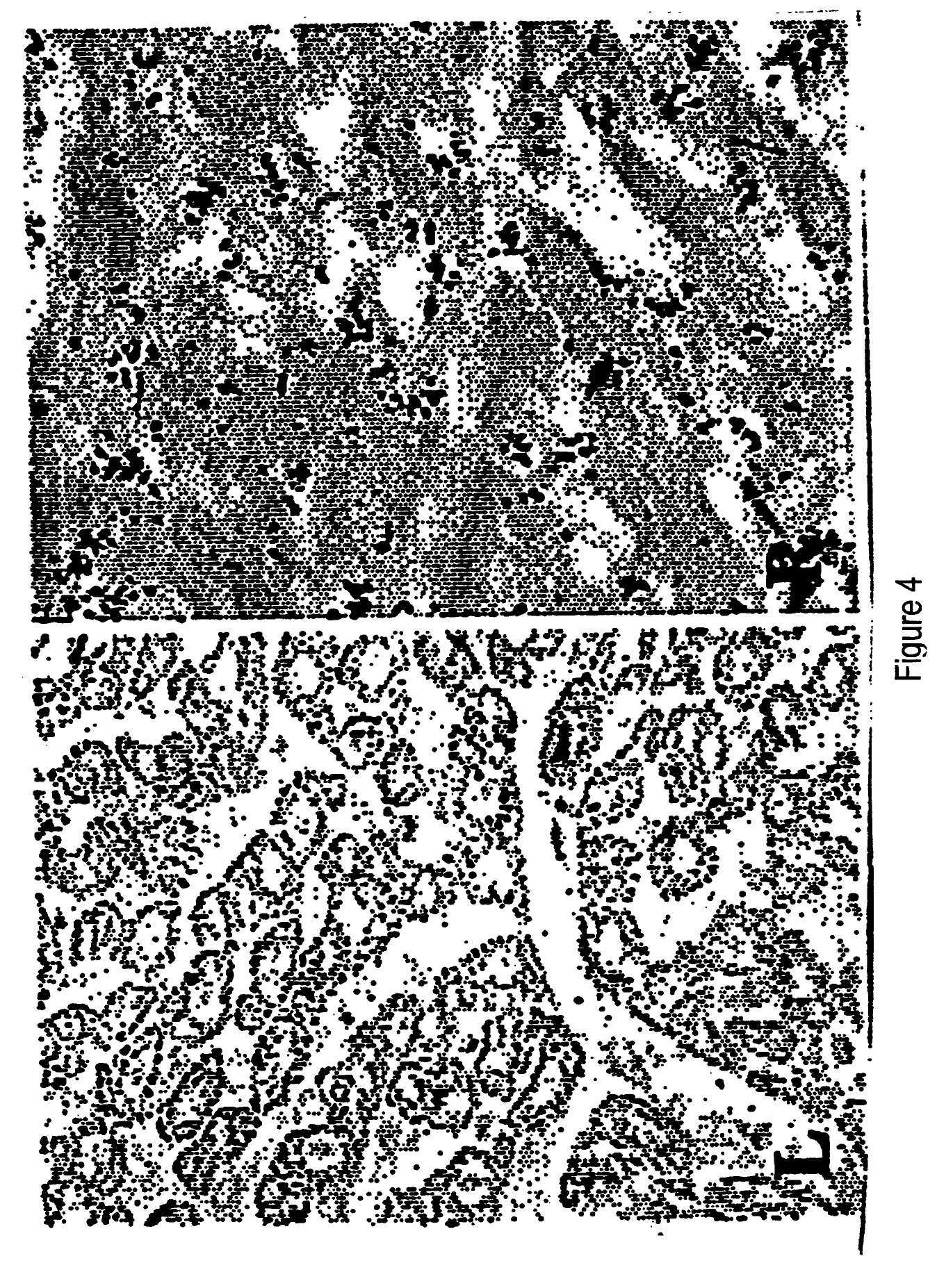

This example illustrates an experiment to evaluate a time course for gene expression after administration of a liposomal gene therapy formulation into the coronary circulation, as provided in example 2 herein. Reporter gene expression was examined at 4, 6, 24 and 48 hours following liposome (1:1 ratio) transfection. FIG. 4 shows a 24 hour and six hour and control carotid artery preparation for prevalence of alkaline phosphatase reporter gene product expression. Gene product expression was seen as early as 6 hours following just a 10 minute exposure to the liposome-reporter gene complex. Expression increased to 24 hours. In another study VCAM-1 (integrin adhesion molecule) expression in transplanted donor hearts was first detected at 6 hours. These data indicate the time course for gene product expression as measured by the gene product of a reporter gene or even a biologic effect of VCAM-1 expression.

### EXAMPLE 4

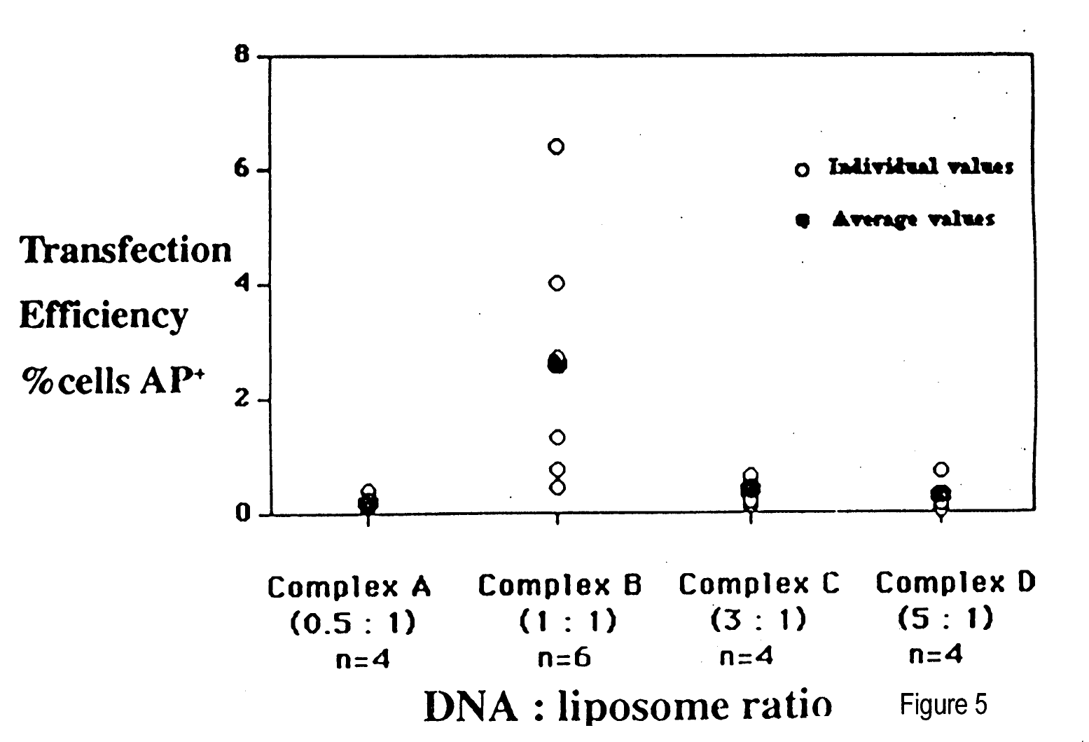

When effecting a reperfusion of ischemic myocardial tissue, it is important to reduce adhesion molecule expression and subsequent pro-inflammatory cytokine cascade in the coronary microvasculature. One means to prevent ischemia-reperfusion injury is to reduce adhesion molecule expression in the coronary microvasculature. This example shows a procedure whereby one can visualize NFκB activity using an immunocytochemistry technique with an antibody specific to a p65 epitope that is unmasked only when p65 is translocated to a cells nucleus. Thirteen rabbit donor hearts were subject to 45 minutes of ischemia followed by 0, 2, 4 or 24 hours of reperfusion. The number of nuclei positive for activated p65 was compared to the total number of nuclei visible in a high-powered field. FIG. 5 shows a comparison between baseline and background activation (no ischemia and no reperfusion) and a positive sample (45 minutes of ischemia and 24 hours of reperfusion). There is a direct relationship between increasing ischemia-reperfusion injury and the extent of NFκB activation shown in Table 1

TABLE 1

| grade | 0 hr | 2 hr | 4 hr | 24 hr |
|---|---|---|---|---|
| 3+ | | | 1 | 2 |
| 2+ | | | 5 | |
| 1+ | 2 | 2 | 1 | |
| 0 | | | | |
| n= | 2 | 2 | 7 | 2 |

The data in Table 1 show the percentage of nuclei with activated NFκB versus increasing ischemia-reperfusion injury. The time 0 hearts have no ischemia or reperfusion. The table shows reperfusion times across the top with ischemia time held constant at 45 minutes. The NFκB nuclei are classified semi-quantitatively as 1+=25%; 2+=50%; 3+=75%; and 4+=>75%. These data show the direct relationship between increasing ischemia-reperfusion injury and the extent of NFκB activation. Similar to adhesion molecule expression, maximal NFκB activation after ischemia-reperfusion injury was seen at 24 hours. Therefore, there is sufficient time for transfected genes to be effective in addressing the ischemia-reperfusion process without the need for instantaneous therapeutic intervention associated with intravenous administration of a small molecule agent.

### EXAMPLE 5

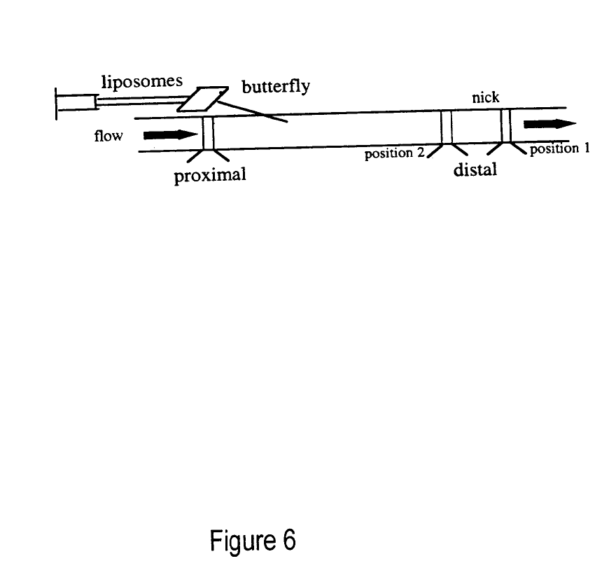

This example illustrates a model wherein reporter gene constructs and VEGF gene constructs transfected into in situ rabbit carotid artery segments expressed the appropriate gene products. An in situ rabbit carotid transfection model without transplantation consists of the isolation of the carotid artery after systemic heparinization. A 4 cm segment of carotid artery was transiently isolated between atraumatic vascular clamps as shown in FIG. 6. Blood was flushed from the segment with D5W, and liposomes containing the gene product as made according to the present invention were instilled through a butterfly catheter (FIG. 6). A distal clamp was moved proximal to a nick, closing the segment (FIG. 6). After a defined exposure time, blood flow was reinstituted by releasing the clamps. The animals were sacrificed 24 hours later to assess the prevalence of reporter gene product activity.

The reporter gene used was CAT. In six rabbits, carotid segments were clamped for either 10 or 30 minutes after the instillation of liposome-CAT DNA complexes. In these segments transiently isolated from blood flow, 10 minutes of contact with the liposomal gene therapy vehicle resulted in gene product expression indistinguishable from that seen at 30 minutes. Therefore, even a 10 minute uptake time was sufficient to see gene product expression using a liposomal gene therapy delivery vehicle to vascular endothelium. Therefore, the foregoing technique is appropriate for routine cardiac procedures, such a balloon angioplasty.

The transfected reporter genes remained localized in the transfected segments even after blood flow was reinstituted. After blood flow was resumed, reporter gene product expression was markedly less in the carotid segment immediately distal (i.e., within 1 cm) to the transfected segment in 5 of the 6 rabbits treated in situ. Moreover, there was minimal expression seen in the systemic end organ (brain). Therefore, these in situ model data indicate that the risk of generalized angiogenesis from local delivery of angiogenic growth factor gene therapy to myocardial tissue is minimal and is highly localized to the vascular bed of delivery.

The duration of gene expression was tested in seven rabbits using the foregoing in situ rabbit carotid transfection model without transplantation. The same transfection parameters were followed except the rabbits were kept alive for 21 days post-transfection to test the duration of gene expression. The reporter gene product expression was measured as CAT activity per milligram of total protein for the transfected carotid artery as compared to the untransfected contralateral carotid artery. At 21 days, 3 of 7 animals still exhibited CAT activity over 2×10⁵ cpm/mg protein, another three animals had 6-10 fold greater gene product expression in the affected carotid versus the contralateral carotid. Therefore, liposome transfection can result out to 21 days of gene product expression using the liposome transfection delivery system delivered to vascular endothelium.

### EXAMPLE 6

This example illustrates an in vitro experiment with cultured rabbit arterial endothelial cells that were transfected with a liposomal DNA complex containing a luciferase reporter gene encoding a luciferase reporter gene product. Two different liposomal formulations were tried, including a DOPSA/DOPE (Gibco) lipid complex or a formulation having a cationic to neutral lipid ratio of about 1:1. The DNA concentration was either 1 or 5 μg/10⁵ cells. The liposomal content to DNA (μg) ratio varied from 20 to 200 μmoles of cationic lipid. The in vitro method allowed for examination of many different combinations and ratios.

The optimal combination was found to be 40 micromoles of cationic lipid per μg DNA overlayed onto 10⁵ cells. Incubation times were also examined to find optimal reporter gene product expression and 4 hours was found to be an optimal time for incubation. However, luciferase activity (reporter gene product expression) was also found with as little as 30 minutes of incubation time. Therefore, these data provide evidence that the inventive method is functional to provide a means for local delivery of angiogenic growth factors to the coronary circulation.

We claim:

1. A method for revascularizing ischemic tissue or improving the vascular flow of ischemic tissue, comprising administering an angiogenesis inducing gene encoding an angiogenesis growth factor encapsulated in a liposomal carrier vehicle into the ischemic tissue.

2. The method of claim 1 wherein a means for administering the angiogenesis inducing gene encoding an angiogenesis growth factor is by retrograde perfusion into the vasculature of the ischemic tissue.

3. The method of claim 1 wherein a means for administering the angiogenesis inducing gene encoding an angiogenesis growth factor is by retrograde perfusion into the coronary circulation.

4. The method of claim 3 wherein the means for administration into the coronary circulation is through retrograde perfusion of the coronary vasculature.

5. The method of claim 1 wherein the means for retrograde perfusion is by a catheter inserted into a patient's regional coronary venous system supplied into that region of ischemic myocardium, wherein the catheter has an inflatable balloon containing the liposomally encapsulated gene vectors.

6. The method of claim 1 wherein, the angiogenesis-inducing gene is selected from the group consisting of VEGF, ICAM-1, FGF, EGF, nitric acid synthase (NOS), E-selectin, and L combinations thereof.

7. The method of claim 1 wherein the liposome containing the angiogenesis-inducing gene has a ratio of lipid to nucleic acid material in the range of from about 1:2 to about 2:1.
